(19) United States
(12) Patent Application Publication
(10) Pub. No.: US 2002/0006942 A1
(43) Pub. Date: Jan. 17, 2002

(54) Methods of treating liver disorders and disorders associated with liver function

(76) Inventor: Roger A. Davis, Solana Beach, CA (US)

Correspondence Address: Robert M. Bedgood, PILLSBURY WINTHROP LLP, 50 Freemont Street, San Francisco, CA, 94105, US

(21) Appl. No.: 09/792,631

(22) Filed: Feb. 23, 2001

Related U.S. Application Data
(60) Provisional application No. 60/184,592, filed on Feb. 24, 2000.
(60) Provisional application No. 60/187,321, filed on Mar. 6, 2000.

(51) Int. Cl. A61K 31/5585; A61K 31/5575; A61K 31/426; A61K 31/20

(52) U.S. Cl. 514/369; 514/573; 514/558; 514/469

(57) ABSTRACT

Methods of treating liver inflammatory condition, disease or disorder are provided. Methods include administering amounts of a PPARγ agonist sufficient to ameliorate the inflammatory condition, disease or disorder. Methods of treating conditions associated with excess or undesirable cholesterol levels or decreased HDL levels or decreased CYP7A expression are also provided. Methods include administering amounts of a PPARγ agonist sufficient to decrease cholesterol levels or increase HDL levels or CYP7A expression.

### STATEMENT AS TO FEDERALLY SPONSORED RESEARCH

The invention was made with Government support from the Heart, Lung and Blood Institute of the National Institutes of Health grant no. HL57974.

### TECHNICAL FIELD

The invention relates to inhibiting production of one or more cytokines in liver, inhibiting bile acid mediated repression of cholesterol-7α-hydroxylase (CYP7A), and reducing physiological symptoms or treating pathological disorders associated with 1011- overproduction/production of liver cytokine or underexpression/repression of cholesterol-7α-hydroxylase (CYP7A).

### BACKGROUND

Bile acids, the major metabolites produced from cholesterol, are amphipathic steroid detergents necessary for the digestion and absorption of fat soluble nutrients from the intestine (Russell, et al., 1992, Biochemistry 31 (20):4737-4749; Vlahcevic, et al., 1992. In Seminars in Liver Disease. Vol. 12. M. A. Rothschild, editor. Thieme Medical Publishers, New York, Stuttgart, 403-419; Edwards, et al., 1996. In New comprehensive Biochemistry. Vol. 31. D. E. Vance, and J. Vance, editors. Elsevier, Amsterdam. 341-362). The conversion of cholesterol to bile acids is regulated by the expression of cholesterol-hydroxylase (CYP7A1) a cytochrome P450 enzyme unique to the liver parenchymal cell (Noshiro, et al., 1990, FEBS Lett. 268:137-140; Jelinek, et al., 1990, J Biol Chem. 265 (14):8190-8197; Li, et al., 1990, J Biol. Chem., 265: 12012-12019.). Bile acid synthesis exhibits negative feedback regulation (Bergstrom, et al., 1958, Acta Physiol. Scand. 43:1-7; Shefer, et al., 1969, J Lipid Res. 10:646-655.) by decreasing the enzymatic activity of CYP7A (Shefer, et al., 1970, J Lipid Res. 11 (5):404-411).

It is generally accepted that bile acids returning to the liver via the enterohepatic circulation repress the transcription of the CYP7A1 gene (Russell, et al., 1992, Biochemistry 31 (20):4737-4749; Vlahcevic, et al., 1992. In Seminars in Liver Disease. Vol. 12. M. A. Rothschild, editor. Thieme Medical Publishers, New York, Stuttgart, 403-419; Edwards, P. A., and R. A. Davis. 1996. In New comprehensive Biochemistry. Vol. 31. D. E. Vance, and J. 1 Vance, editors. Elsevier, Amsterdam. 341-362.). Bile acid negative feedback repression of CYP7A1 has been experimentally demonstrated by infusing bile acids into bile fistulae rats (Pandak, et al., 1991, J. Biol. Chem. 266:3416-3421) and hamsters (Spady, et al., 1996. J Biol. Chem. 271:18623-18631). The ability of different bile acids to repress CYP7A1 correlates with the hydrophobic index of the bile acid infused: chenodeoxycholic acid (CDCA) is a potent repressor, whereas ursodeoxycholic acid (UDCA) does not repress (Heuman, et al., 1989, J. Lipid Res. 30:1161-1171.). Bile acid repression of CYP7A1 has been demonstrated using primary cultured rat hepatocytes (Stravitz, 1993, J. Biol. Chem. 268 (19):13987-13993) and human hepatoma HepG2 cells (Crestani, et al., 1994, Biochem Biophys Res Commam. 198 (2):546-553; Taniguchiet al., 1994, J. Biol. Chem. 269:10071-10078; Makishima, et al., 1999, Science. 284 (5418):1362-1365), but not in a differentiated line of rat hepatoma L35 cells (Trawick, et al., 1996, J. Lipid Res. 37:24169-24176; Trawick, J. D., et al., 1997, J. Biol. Chem. 272:3099-3102). The level of expression of CYP7A1 by L35 cells is similar to that of rat liver and it varies in response to essentially all hormones, cytokines, and effectors reported to alter CYP7A1 expression in rat liver, (Trawick, et al., 1996, J. Lipid Res. 37:24169-24176; Trawick, et al., 1997, J. Biol. Chem. 272:3099-3102.

An inbred mouse strain (C3H/HeJ) has been described that like L35 cells, displays resistance to bile acid repression of CYP7A1 (Dueland, et al., 1993, J. Lipid Res. 34:923-931; Dueland et al., 1997, J. Lipid Res. 38:1445-1453; Machleder, et al., 1997, J. Clin Invest. 99 (6):1406-1419). C3H/HeJ mice are also resistant to diet-induced atherosclerosis, whereas C57BL/6 mice are susceptible (Paigen, et al., 1987, Proc Natl Acad Sci USA. 84 (11):3763-3767; Liao, et al., 1993, J Clin Invest. 91 (6):2572-2579; Liao, et al., 1994, J Clin Invest. 94 (2):877884; Berliner, et al., 1995, Circulation. 91:2488-2496; Shih, et al., 1995, Mol Med Today. 1 (8):364-372). Strain specific susceptibility to diet-induced atherosclerosis has been linked to hepatic inflammation (Liao, et al., 1993, J Clin Invest. 91 (6):2572-2579; Liao, et al., 1994, J Clin Invest. 94 (2):877-884), repression of CYP7A1 (Dueland, et al., 1993, J. Lipid Res. 34:923931; Machleder, et al., 1997, J Clin Invest. 99 (6):1406-1419) and a concomitant and parallel reduction in plasma HDL (Dueland, et al., 1997, J. Lipid Res. 38:1445-1453; Machleder, et al., 1997, J Clin Invest. 99 (6):1406-1419; Shih, et al., 1996, J Clin Invest. 97 (7):1630-1639).

### SUMMARY OF THE INVENTION

The present invention provides methods of inhibiting production of one or more cytokines by a cell of the liver. In one embodiment, a method includes contacting a cell of the liver that expresses a cytokine with an amount of a PPARγ agonist or agent that increases expression of PPARγ sufficient to inhibit production of a cytokine by the cell. In various aspects, the cytokine comprises an inflammatory cytokine (e.g., IL-1α, IL-1β, TNFα, IFNβ, IFNγ or TGF-β1). In various additional aspects, the cell is a Kupffer cell, hepatocyte, bile ductal cell, parenchymal cell, ito cell, stellate cell, portal or central vein cell, epithelial cell or endothelial cell. In another aspect, the PPARγ agonist comprises rosiglitazone, or an analogue or derivative thereof. In another aspect, the PPARγ agonist comprises a thiazolidinedione (e.g., pioglitazone, darglitazone, fluoroglitazone, troglitazone, BRL 49653, ciglitazone, englitazone, AD 5075, a salt thereof or an analogue or derivative thereof). In still another aspect, the PPARγ agonist comprises a prostaglandin, a fatty acid or a metabolite thereof. Methods include contacting in vitro, in vivo and ex vivo.

Accordingly, the invention also provides methods of inhibiting production of a cytokine in the liver of a subject. In one embodiment, a method includes administering a PPARγ agonist or agent that increases expression of PPARγ to the subject in an amount sufficient to decrease production of one or more cytokines in the liver. In various aspects, the cytokine comprises an inflammatory cytokine (e.g., IL-1α, IL-1β, TNFα, IFNβ, IFNγ or TGF-β1). In another aspect, the PPARγ agonist comprises rosiglitazone, or an analogue or derivative thereof. In another aspect, the PPARγ agonist comprises a thiazolidinedione (e.g., pioglitazone, darglitazone, fluoroglitazone, troglitazone, BRL 49653, ciglitazone, englitazone, AD 5075, a salt thereof or an analogue or derivative thereof). In still another aspect, the PPARγ agonist comprises a prostaglandin, a fatty acid or a metabolite thereof. Subjects useful in the methods include a human.

Cytokine production is associated with and causes various disorders and pathological conditions. Thus, methods additionally include inhibiting production of a cytokine in liver in order to treat the disorder. Any disorder in which cytokines play a role can therefore be treated by a method of the invention.

Accordingly, the invention also provides methods of inhibiting liver damage or susceptibility to liver damage caused by production of a cytokine in the liver. The invention additionally provides methods of treating or reducing the risk of an inflammatory condition of the liver in a subject. The methods include administering a PPARγ agonist or agent that increases expression of PPARγ to the subject in an amount sufficient to treat or reduce the risk of the inflammatory condition of the liver, or administering a PPARγ agonist or agent that increases expression of PPARγ to a subject in an amount sufficient to inhibit liver damage or susceptibility to liver damage caused by production of the cytokine, respectively.

Inflammatory conditions treatable include, but are not limited to, alcoholic liver disease, cirrhosis, tylenol poisoning, Reye's syndrome, acute or chronic xenobiotic poisoning, acute or chronic hepatitis infection, or cholestatic liver disease.

The invention also provides methods of increasing expression of cholesterol-7α-hydroxylase or inhibitng bile-acid mediated repression of CYP7A. In one embodiment, a method includes contacting a cell of the liver with an amount of a PPARγ agonist sufficient to increase CYP7A expression. In another embodiemnt, a method includes contacting a cell of the liver with an amount of a PPARγ agonist sufficient to inhibit bile-acid mediated CYP7A repression. Methods include contacting in vitro, in vivo and ex vivo, e.g., in a subject such as a human.

Cholesterol production is associated with and causes various disorders and pathological conditions. Thus, methods additionally include inhibiting or decreasing cholesterol, LDL or VLDL, or increasing HDL in order to treat the disorder or reduce the risk or susceptibility to the disorder. Any disorder in which excess or undesirable cholesterol or decreased HDL play a role can therefore be treated or have its risk of occurrence reduced by a method of the invention.

Accordingly, the invention provides methods of decreasing low density lipoprotein (LDL), VLDL or cholesterol in a subject. In one embodiment, a method includes administering a PPARγ agonist or agent that increases expression of PPARγ to the subject in an amount sufficient to decrease low density lipoprotein (LDL), VLDL or cholesterol. In one aspect, the PPARγ agonist comprises rosiglitazone or an analogue or derivative thereof. In another aspect, the PPARγ agonist comprises a thiazolidinedione (e.g., pioglitazone, darglitazone, fluoroglitazone, troglitazone, BRL 49653, ciglitazone, englitazone, AD 5075, a salt thereof or an analogue or derivative thereof). In yet another aspect, the PPARγ agonist comprises a prostaglandin, a fatty acid or a metabolite thereof. Subjects useful in the methods include a human.

The invention also provides methods of increasing high density lipoprotein (HDL) in a subject. In one embodiment, a method includes administering a PPARγ agonist or agent that increases expression of PPARγ to the subject in an amount sufficient to increase high density lipoprotein (HDL). In one aspect, the PPARγ agonist comprises rosiglitazone or an analogue or derivative thereof. In another aspect, the PPARγ agonist comprises a thiazolidinedione (e.g., pioglitazone, darglitazone, fluoroglitazone, troglitazone, BRL 49653, ciglitazone, englitazone, AD 5075, a salt thereof or an analogue or derivative thereof). In yet another aspect, the PPARγ agonist comprises a prostaglandin, a fatty acid or a metabolite thereof.

Specific disorders associated with undesirable or excess levels of cholesterol, LDL or VLDL, or reduced levels of HDL cholesterol include artherosclerotic lesions leading to artherosclerosis, coronary heart disease, cardiac ischemia, stroke, or hypertension, peripheral vascular disease or dyslipidemia.

Thus, the invention provides methods of reducing artherosclerosis or coronary heart disease, or susceptibility to artherosclerosis or coronary heart disease in a subject having or at risk of having artherosclerosis or coronary heart disease. The invention also provides methods of reducing the risk of heart attack or angina in a subject. In various embodiments, a method includes administering a PPARγ agonist or agent that increases expression of PPARγ to the subject in an amount sufficient to reduce artherosclerosis or coronary heart disease, or susceptibility to artherosclerosis or coronary heart disease, or administering a PPARγ agonist or agent that increases expression of PPARγ to the subject in an amount sufficient to decrease the risk of heart attack or angina, respectively.

PPARγ antagonists or agents decreasing expression of PPARγ can be used to increase production of a cytokine in a cell of the liver. Thus, the invention provides methods of increasing production of a cytokine in a cell of the liver. In one embodiment, a method includes contacting a cell of the liver that expresses a cytokine with a sufficient amount of a PPARγ antagonist or an agent that decreases expression of PPARγ to increase production of the cytokine by the liver cell. In various aspects, the cytokine comprises an inflammatory cytokine (e.g., IL-1α, IL-1β, TNFα, IFNβ, IFNγ or TGF-β1). In additional aspects, the liver cell is a Kupffer cell or hepatocyte. Methods include contacting in vitro, in vivo and ex vivo, e.g., in a subject such as a human.

PPARγ antagonists or agents decreasing expression of PPARγ can be used to decrease cholesterol-7α-hydroxylase (CYP7A) expression or increase bile acid mediated repression of CYP7A. Thus, the invention provides methods of decreasing CYP7A expression and increasing bile acid mediated repression of CYP7A. In one embodiment, a method includes contacting a cell of the liver with an amount of a PPARγ antagonist sufficient to decrease CYP7A expression by the cell. In various aspects, the cell is a Kupffer cell, hepatocyte, bile ductal cell, parenchymal cell, ito cell, stellate cell, portal or central vein cell, epithelial cell or endothelial cell. Methods include contacting in vitro, in vivo and ex vivo, e.g., in a subject such as a human.

In addition, cytokines can be used to inhibit bile acid production. Thus, the invention further provides methods of inhibiting bile acid production by increasing production of a cytokine. In one embodiment, a method includes contacting a cell of the liver with an amount of a PPARγ antagonist or an agent that decreases expression of PPARγ, or a cytokine in an amount sufficient to inhibit bile acid production. In various aspects, the cytokine comprises an inflammatory cytokine (e.g., IL-1α, IL-1β, TNFα, IFNβ, IFNγ or TGF-β1). In additional aspects, the liver cell is a Kupffer cell or hepatocyte. Methods include contacting in vitro, in vivo and ex vivo, e.g., in a subject such as a human.

### DESCRIPTION OF DRAWINGS

FIG. 1 shows that a bile acid-containing atherogenic diet decreases CYP7A1 MRNA expression and increases hepatic cytokine MRNA expression in C57BL/6 mice but not in C3H/HeJ mice. C57BL/6J and C3H/HeJ mice were fed normal chow or bile acid-containing atherogenic diet and liver RNA expression determined for (A) CYP7A1 and GAPDH and (B) the indicated cytokine.

FIG. 2 shows that expression of cytokine mRNAs by THP-1 cells correlates with the ability of conditioned medium to repress CYP7A1 mRNA expression by rat hepatoma L35 cells. (A) CDCA requires THP-1 cells in order to repress CYP7A1 expression by L35 cells; (B) CDCA but not UDCA induces the expression of cytokine mRNA by THP-1 cells via a process that is blocked by the PPARγ agonist rosiglitazone. THP-1 cells treated with 0.1% BSA (lanes 1 & 5), 0.1% BSA containing 100 μM CDCA (lanes 2 & 6), 0.1% BSA containing 100 μM UDCA (lanes 3 & 7) or 0.1% BSA containing 100 μM CDCA and 500 nM rosiglitazone (lanes 4 & 8); RR(C) PPARγ agonist rosiglitazone blocks CDCA induced production by THP-1 cells of conditioned medium which represses the expression of CYP7A1 mRNA by L35 cells. Values represent the mean of duplicate plates of cells; (D) ThNFa. represses expression of CYP7A1 nmRNA by L35 cells. Human TNFa concentrations are indicated and each mRNA value represents the level of CYP7A mRNA to β-actin mRNA as the mean ±S.D of three replicate plates of cells.

FIG. 3 shows that CDCA inactivates LXR transcription while activating FXR transcription. L35 cells transiently transfected with either a (A) FXR-luciferase reporter or (B) a LXR-luciferase reporter with a CMV-driven expression plasmid encoding FXR or LXR, as indicated. CDCA was added where indicated and relative luciferase activity is presented as the mean ±S.D of three replicate plates of cells as the ordinate in a log form.

FIG. 4 shows that PPARγ agonist rosiglitazone blocks repression of CYP7A1 mRNA as well as the decrease in HDL cholesterol in animals caused by bile acids. (A) The relative content of rat CYP7A1 mRNA compared to GAPDH in female C57BL/6J mice fed either chow or the bile acid-containing atherogenic diet; (B) Plasma HDL cholesterol levels were determined from blood obtained from the mice. Each value represents the mean ±S.D of six separate mice. *Denotes a significant difference between the values for the rosiglitazone treated chow-fed mice and the rosiglitazone mice fed the bile acid-containing atherogenic diet, p<0.01.

FIG. 5 shows (A) hepatic expression of CYP7A1 transgene mRNA (stippled bars) and non-transgenic control mice (open bars) fed chow or a “high-fat” diet containing taurocholate; (B) CYP7A1 enzyme activity in hepatic microsomes. The mean ±SD of the relative levels of expression of hepatic CYP7A1 MRNA relative to GAPDH is shown for 5 mice in each diet group. The activity of CYP7A1 was determined using hepatic microsomes obtained from 5 mice in each diet group and is expressed as the mean ±SD. The differences between CYP7A1 and non-transgenic littermates were statistically significant, p<0.01.

FIG. 6 shows plasma (A) VLDL, IDL and LDL cholesterol levels and (B) HDL cholesterol levels in CYP7A1 transgenic and non-transgenic mice (6 per group) fed a chow or high fat diet. Assays were done in quadruplicate. The differences between CYP7A1 and nontransgenic littermates were statistically significant, p<0.01.

FIG. 7 shows that CYP7A1 transgene expression prevents artherosclerotic lesions in the aortic sinus of mice.

### DETAILED DESCRIPTION OF THE INVENTION

The invention is based, in part, on the discovery of the relationship between bile acids and cytokine production in the liver. Excess bile acid production increases expression of liver cytokines. The invention is also based, in part, on the discovery of the relationship between cytokine production in liver and regulation of cholesterol-7α-hydroxylase (CYP7A) expression. Liver cytokines decrease expression of CYP7A. Peroxisome proliferator activated-y receptor (PPARS) regulates these processes, that is, PPARγ agonists decrease production of one or more cytokines in the liver and inhibit repression of CYP7A expression. Thus, compounds that increase or stimulate PPARγ expression (transcription or translation) or activity, such as agents or proteins that increase or stimulate PPARγ expression, or ligands that increase or stimulate PPARγ activity (i.e., agonists), are useful for inhibiting or preventing production of one or more cytokines in liver, increasing expression of CYP7A or inhibiting or preventing repression of CYP7A mediated by bile acids. Compounds that inhibit or prevent PPARγ expression or activity, such as agents or proteins that inhibit PPARγ expression, or ligands that reduce or block PPARγ activity (i.e., antagonists), are useful for increasing or stimulating production of one or more cytokines in liver, for decreasing CYP7A expression or for increasing or stimulating bile acid mediated repression of CYP7A expression. Such PPARγ expression or activity stimulating and inhibiting compounds are additionally useful in therapeutic protocols including treating a subject in order to inhibit or increase cytokine production, or inhibit or increase CYP7A expression.

Thus, in accordance with the invention, there are provided methods of inhibiting, reducing or preventing production of one or more cytokines in a cell of the liver. In one embodiment, a method includes contacting a cell of the liver that expresses or is capable of expressing a cytokine with an amount of a PPARγ agonist sufficient to inhibit production of a cytokine by the cell. In one aspect, the cytokine is an inflammatory cytokine, e.g., IL-1α, IL-1β, TNFα, IFNβ, IFNγ or TGF β1. In another aspect, the cell of the liver is a Kupffer cell or hepatocyte. In yet another aspect, the PPARγ agonist comprises a thiazolidinedione, such as rosiglitazone, pioglitazone, darglitazone, fluoroglitazone, troglitazone, BRL 49653, ciglitazone, englitazone, AD 5075, a salt thereof or an analogue or derivative thereof. In additional aspects, a method includes contacting in vitro, in vivo (e.g., in a subject such as a human) or ex vivo.

In accordance with the invention, there are also provided methods for increasing CYP7A expression and for inhibiting or preventing bile acid mediated repression of CYP7A expression. In one embodiment, a method includes contacting a cell of the liver with an amount of a PPARγ agonist sufficient to increase cholesterol-7α-hydroxylase (CYP7A) expression. In another embodiment, a method of the invention includes contacting a cell of the liver with an amount of a PPARγ agonist sufficient to reduce bile-acid mediated cholesterol-7α-hydroxylase (CYP7A) repression by the cell. In one aspect, the cell of the liver is a Kupffer cell or hepatocyte. In another aspect, the PPARγ agonist comprises a thiazolidinedione, such as rosiglitazone, pioglitazone, darglitazone, fluoroglitazone, troglitazone, BRL 49653, ciglitazone, englitazone, AD 5075, a salt thereof or an analogue or derivative thereof. In yet other aspects, a method includes contacting in vitro, in vivo (e.g., in a subject such as a human) or ex vivo.

As used herein, the term “cytokine” means a molecule that modulates immune response, either increasing (e.g., immune stimulating) or decreasing (e.g., immune tolerizing) immune response. Cytokines include molecules that participate in regulation of either cell mediated immunity (e.g., regulating cell chemotaxis, proliferation, differentiation, activity, secretion of other molecules such as cytokines, etc.), or humoral immunity (increasing antibody production). An “inflammatory cytokine” mediates or contributes to an immune response that directly or indirectly causes localized (e.g., a tissue, organ or region of a subject) or systemic (hypersensitivity, anaphylaxis) inflammation. Specific examples of cytokines include, but are not limited to, interleukins, such as IL-1α, IL-1β, IL-2 through IL-23; interferons, such as IFNα, IFNβ, IFNγ; and TNFα and TGF β1.

The finding that a PPARγ agonist inhibits activation of various liver cytokines indicates that a cell of the liver contains one or more factors that respond to a PPARγ agonist, such as PPARγ, and also is capable of expressing one or more cytokines. As used herein, the term “cell of the liver” means a cell that normally resides within the tissue encompassed by the liver capsule. Liver cells therefore include, for example, hepatocytes, parenchymal cells, ito cells, stellate cells, bile duct cells, epithelial cells and endothelial cells, as well as Kupffer cells, which are macrophages/monocytes that reside in the liver. Stem cells and progenitor cells in the liver are also included in the meaning of the term. A “stem cell” or “progenitor cell” means a cell that can give rise to phenotypically and genotypically identical daughters or differentiate into one or more different cell types including a final cell type (e.g., a terminally differentiated cell).

A cell of the liver therefore does not include peripheral monocytes or macrophages circulating in the blood stream. Unlike peripheral macrophages/monocytes which are replaced within about three months in the circulation, replacement of Kupffer cells occurs very slowly; in one study after one year, only 50% of the cells had been replaced from the bone marrow (Kennedy, et al., 1997, Blood 90 (3):986-993).

Cells of the liver therefore include cells that express a cytokine or are capable of expressing a cytokine in a fashion regulatable by a PPARγ agonist even if the cell lineage is distinct from the immune system. For example, hepatocytes, bile duct cells, biliary epithelial cells, central vein cells and endothelial cells all have been reported to produce TNFα. A cell that may not be actively expressing a cytokine under certain conditions but does express a cytokine whose expression is influenced by a PPARγ agonist is also included as preventing or inhibiting production of a cytokine by these cells is useful in the methods of the invention. Cells that express cytokines can be identified by fractionating liver cells and analyzing for the presence of cytokines. For example, fluorescent activated cell sorting (FACS) can separate different cell types and RNA or protein from the differentially sorted cells can be extracted and analyzed for cytokine expression using northern blotting, western blotting immunoprecipitation, etc.

As used herein, the term “PPARγ agonist” means a molecule that decreases or prevents production of one or more cytokines, increases CYP7A expression, or inhibits or prevents bile acid mediated repression of CYP7A expression. A “PPARγ antagonist” means a molecule that increases or stimulates production of a cytokine, decreases CYP7A expression, or increases bile acid mediated repression of CYP7A expression. Agonists and antagonists can essentially be any organic or inorganic molecule having the requisite activity. Exemplary forms include small organic molecules, such as fatty acids, lipids, triglycerides, carbohydrates or sugars, protein, nucleic acid and metabolites thereof.

Although it is believed that a PPARγ agonist or antagonist effects its cytokine production and CYP7A expression regulatory function by increasing or decreasing activity of PPARγ, respectively, it is possible that a PPARγ agonist or antagonist may act through an effector molecule distinct from or in addition to PPARγ to regulate cytokine production or CYP7A expression. Accordingly, the invention methods do not preclude PPARγ agonists and antagonists that act entirely or in part with one or more effector molecules distinct from PPARY to modulate cytokine production or CYP7A expression.

PPARγ agonists include natural compounds present in the in vivo environment and synthetic compounds, such as drugs. Specific examples of natural compounds include fatty acids and fatty acid metabolites, eicosanoids and prostaglandins, such as prostaglandin J₂ (PGJ₂) or prostaglandin D₂ (PGD₂). Specific examples of fatty acid metabolites include fatty acids modified by oxygenase enzymes, for example, U-oxidized forms of fatty acid. Specific examples of synthetic compounds include drugs used to treat diabetes, which include, for example, thiazolidinediones. Specific examples of thiazolidinediones are pioglitazone, darglitazone, fluoroglitazone, troglitazone, BRL 49653, ciglitazone, englitazone, AD 5075 and Rezulin. These are but a few specific examples of PPARγ agonists applicable in the methods of the invention. Additional agonists and antagonists are known in the art, or can be identified by screening test compounds for agonist or antagonist activity using PPARγ activity assays disclosed herein or known in the art.

PPARγ agonists and antagonists also include analogues or derivatives. As used herein, the term “analogue” means a structurally similar molecule that has at least part of the function of the comparison molecule. In other words, the analogue would still retain at least a part of the activity of the comparison molecule. Thus, an analogue of a PPARγ agonist would be a structurally similar molecule that increases or stimulates PPARγ activity. An example of a rosiglitazone analogue is a molecule with the same number of carbons in the backbone, but has one or more side chains removed, replaced or otherwise altered, e.g., an alcohol converted to an enol group, a methyl converted to an ethyl group, or vice versa, etc. A particular example of a prostaglandin analogue is prostaglandin J₂ (PGJ₂) analogs (e.g., ¹²-prostaglandin 32 and 15-deoxy-Δ^{12,14}-prostaglandin J₂).

As used herein, the term “derivative” means a modified form of the molecule, that is, the molecule is chemically or otherwise modified in comparison to the original form. Again, the derivative would still retain at least a part of the activity of the unmodified molecule. Thus, a derivative of a PPARγ agonist would be a modified form of an agonist molecule that increases or stimulates PPARγ activity. Particular examples of derivatives include fibric acid derivatives of PGJ₂. Thus, PPARγ agonists and antagonists include analogues or derivatives of rosiglitazone, pioglitazone, darglitazone, fluoroglitazone, troglitazone, BRL 49653, ciglitazone, englitazone, AD 5075 and Rezulin.

PPARγ agonists and antagonists also include proteins, polypeptides or peptides that bind to and modulate a PPARγ activity. For example, an antibody or fragment thereof that specifically binds to PPARγ ligand binding domain may sterically interfere with binding of ligand agonist and, therefore, inhibit a PPARγ activity. Alternatively, antibody binding may stimulate a PPARγ activity because the antibody mimics a ligand agonist or induces a conformational change in PPARγ that stimulates a PPARγ activity. Thus, a protein, polypeptide or peptide that binds to PPARγ and increases activity (an agonist) can be used to inhibit cytokine production or inhibit bile acid mediated repression of CYP7A expression; and a protein, polypeptide or peptide that binds to PPARγ and decreases activity (an antagonist) can be used to increase or stimulate cytokine production or increase bile acid mediated repression of CYP7A expression.

PPARγ agonists and antagonists can be physically linked or combined with functionally distinct entities that confer a function or activity. As used herein, the term “heterologous functional moiety” means a an entity that imparts a distinct or complementary activity upon another entity when linked or combined with the other entity. Heterologous functional moieties therefore include entities that confer cell (e.g. liver cell) targeting, facilitates cell entry of the molecule or confers regulation of PPARγ activity or has PPARγ agonist or antagonist activity.

Particular examples heterologous functional moieties include proteins or small organic molecules. Specific examples of targeting molecules include an antibody or ligand to a cell surface protein, a natural or engineered vial protein that binds to a cell surface receptor (e.g., a retroviral protein such as HIV tat protein), or a tissue or organ homing molecule (e.g., . Heterologous functional moieties that complement PPARγ agonist or antagonist function include drugs or proteins that modulate cytokine production. A specific example of such a combination is a PPARγ antagonist and a cytokine antisense which together inhibit cytokine production in a cell.

As disclosed herein, PPARγ activity can regulate cytokine production in liver and modulate bile acid mediated repression of CYP7A expression. Thus, increasing or decreasing expression of PPARγ can be used to inhibit or increase or decreases cytokine production in liver, respectively, or inhibit or increase bile acid mediated repression of CYP7A expression, respectively.

Thus, in accordance with the invention, also provided are methods of inhibiting or increasing production of one or more cytokines in a cell of the liver, methods for increasing expression of CYP7A expression and methods for inhibiting bile acid mediated repression of CYP7A expression by modulating expression of PPARγ. In one embodiment, a method includes contacting a cell of the liver that expresses or is capable of expressing a cytokine with a sufficient amount of an agent that increases PPARγ expression to inhibit production of a cytokine by the cell. In another embodiment, a method includes contacting a cell of the liver with a sufficient amount of an agent that increases PPARγ expression to inhibit bile acid mediated repression of CYP7A expression. In other embodiments, a method includes contacting a cell of the liver with a sufficient amount of an agent that decreases PPARγ expression to increase or stimulate production of a cytokine by the cell, or bile acid mediated repression of CYP7A expression.

Cell culture assays using a PPARγ responsive reporter gene can be used to identify PPARγ activity and, therefore, a PPARγ agonist or antagonist. PPARγ expression levels and, therefore, increases or decrease in PPARγ expression can be detected using assays well known in the art including, for example, northern blotting, western blotting, immunoprecipitation, etc.

Cytokine production has been associated with acute or chronic liver inflammation, which can lead to tissue damage in animals. For example, liver insult by a toxin or pathogen can lead to inflammation due, at least in part, to production of cytokines. As disclosed herein, bile acids stimulate cytokine production in liver of animals, e.g., IL-1α, IL-1β, TNFα, IFNβ, IFNγ or TGF-β1 and a PPARγ agonist such as rosiglitazone inhibits production of cytokines. The invention methods, including methods of decreasing or preventing production of a cytokine in a cell of the liver, are therefore applicable in animal subjects, including humans. For example, the methods are useful for inhibiting liver inflammation or damage, or reducing susceptibility to liver inflammation or damage, caused entirely, or at least in part, by production of one or more cytokines in the liver.

Thus, in accordance with the invention, there are provided methods of inhibiting production of a cytokine in the liver of subject. In one embodiment, a method includes administering a PPARγ agonist to the subject in an amount sufficient to decrease production of one or more cytokines in the liver of the subject. In one aspect, a method includes administering a PPARγ agonist to a subject in an amount sufficient to inhibit liver damage or susceptibility to liver damage. In another aspect, a method includes administering a PPARγ agonist to the subject in an amount sufficient to treat or reduce the risk of an inflammatory condition of the liver. In specific aspects, the inflammatory condition of the liver comprises alcoholic liver disease, cirrhosis, Reye's syndrome, acute or chronic toxin exposure (e.g., xenobiotic chemical poisoning such as drugs like tylenol or chenodeoxycholic acid, or drugs used in organ or tissue transplants, substances present in the environment such as carbon tetrachloride, carbon dichloride and chloroform, etc.), acute or chronic hepatitis (e.g., hepatitis A to E) exposure, and cholestatic liver disease. In additional aspects, a condition of the liver comprises jaundice, fatty liver, graft vs. host disease (e.g., transplanted liver rejection), necrosis and hypertension (e.g., portal hypertension).

Trauma associated with liver section removal causes the liver to produce one or more cytokines, which can lead to damage or inflammation of the transplanted liver. Cytokine production, in particular TNFα, is therefore likely to occur when liver sections are removed, such as for a biopsy or surgical resection of a cancer. Thus, in accordance with the invention, there are provided methods of inhibiting cytokine production in liver of subject in which trauma to the liver occurs due to liver removal.

In addition, a transplanted liver may exhibit increased production of cytokines due to trauma associated with the removal and transplantation of the organ and liver hypoxia. Cytokine production by the transplanted liver may promote liver rejection due to stimulation of the subject's immune response. Thus, in accordance with the invention, there are provided methods of inhibiting cytokine production in a transplanted liver of subject in order to reduce or inhibit immune response or rejection of the transplanted liver. In one embodiment, a method includes administering a PPARγ agonist to the subject in an amount sufficient to decrease cytokine production in order to decrease or prevent an immune response directed against the transplanted liver.

As used herein, the term “transplant,” “transplantation” and grammatical variations thereof means a cell, tissue or organ used in grafting, implanting, or transplanting from one part of the body to another part, or from one individual to another individual. The term also includes genetically modified cells tissue and organs, e.g., by ex vivo gene therapy. Additionally, transfer of a tissue from one part of the body to another, or the transfer of tissue from one individual to another also is included.

Furthermore, a subjects' liver may also produce cytokines that participate in the rejection of another tissue transplanted into the host, such as heart, lung, kidney, blood vessels, etc. Thus, in accordance with the invention, also provided are methods of inhibiting production of a cytokine in liver of subject in order to inhibit rejection of a transplanted tissue or organ in the subject.

Excess plasma cholesterol is associated with many disorders including artherosclerosis, causing vasoconstriction of blood vessels levels thereby restricting blood flow to organs, such as the heart. Plasma cholesterol in excess of 200 mg/ml is considered a risk factor for developing coronary heart disease, and increasing the risk of stroke. Expression of CYP7A is important for metabolizing cholesterol into bile acids for elimination, which in turn reduces levels of plasma cholesterol.

As disclosed herein, a PPARγ agonist also inhibits bile acid mediated repression of CYP7A expression in liver of animals. A PPAR₇ agonist additionally increases HDL levels in animals (see, e.g., Example 4). The increase is HDL is likely due, at least in part, to the decease in plasma LDL-cholesterol which in turn is caused, at least in part, by increased CYP7A expression. As disclosed herein, decreased expression of CYP7A is associated with development of artherosclerotic lesion formation and a PPAR7 agonist such as rosiglitazone prevents or inhibits artherosclerotic lesion formation presumably due to increased CYP7A expression leading to decreased levels of LDL or increased levels of HDL alone, or in combination. Thus, a PPARγ antagonist is useful for inhibiting bile acid mediated repression of CYP7A expression, for increasing CYP7A expression, for reducing LDL-cholesterol, for increasing levels of HDL and for decreasing or inhibiting formation artherosclerotic lesions in animal subjects, including humans.

In accordance with the invention, there are provided methods of increasing CYP7A expression, methods of inhibiting bile acid mediated repression of CYP7A expression, methods of reducing LDL-cholesterol, methods of increasing HDL levels and methods of decreasing or inhibiting formation artherosclerotic lesions, including methods of treating disorders resulting from or at increased risk of formation artherosclerotic lesions. In the various methods, a method includes administering a PPARγ agonist to the subject in an amount sufficient to increase CYP7A expression in the subject, to inhibit or decrease bile acid mediated repression of CYP7A expression in the liver, or to reduce LDL-cholesterol levels in the subject. In still another embodiment, a method includes administering a PPARγ agonist to the subject in an amount sufficient to increase HDL levels in the subject.

In a further embodiment, a method includes administering a PPARγ agonist to the subject in an amount sufficient decrease or inhibit formation artherosclerotic lesions, or reduce the risk of artherosclerosis. In one aspect of this embodiment, the rate of artherosclerotic lesion formation is decreased or slowed over time. In an additional aspect the severity of artherosclerosis is decreased or slowed over time. The presence and severity of artherosclerosis and lesion formation can be determined using an angiogram, which detects changes in vessel lumen thickness by injecting a visualizing agent, such as a dye, into the vessel.

In yet a further embodiment, a method includes administering a PPARγ agonist to the subject in an amount sufficient to reduce artherosclerosis or coronary heart disease, or susceptibility to artherosclerosis or coronary heart disease in a subject having or at risk of having artherosclerosis or coronary heart disease. In still another embodiment, a method includes administering a PPARγ agonist to the subject in an amount sufficient to reduce the risk of heart attack or angina in the subject.

PPARγ agonists useful in these and the other methods disclosed herein include, but are not limited to, for example, a thiazolidinedione such as rosiglitazone pioglitazone, darglitazone, fluoroglitazone, troglitazone, BRL 49653, ciglitazone, englitazone, AD 5075, Retulin, and salts, analogues and derivatives thereof. Additional PPARγ agonists known in the art are applicable in the methods of the invention and include, for example, fatty acids, prostaglandins, such as prostaglandin J₂ and prostaglandin D₂, analogues, derivatives and metabolites thereof.

The methods of the invention for treating a subject are applicable for prophylaxis to prevent a condition in a subject. For example, preventing or inhibiting cytokine production in a subject that does not exhibit symptoms of liver inflammation or tissue damage caused by or associated with liver damage, or preventing or inhibiting excess cholesterol levels in a subject that do not yet exhibit increased levels of cholesterol. The methods of the invention also can follow, precede or be used in combination with other therapies including, for example, therapies for reducing liver inflammation (e.g., corticosteroid treatment) or infection (e.g., antivirals to treat hepatitis), reducing cholesterol (e.g., drug therapy such as lipitor) or triglycerides (e.g., drug therapy such as gymfibrizol) or reducing artherosclerosis (e.g., angioplasty or bypass surgery), surgical resection, transplantation, radiotherapy, etc. The skilled artisan can readily ascertain therapies that may be used in a therapeutic regimen in combination with the methods of the invention.

As used herein, the term “subject” means an animal which express cytokines or CYP7A, or bile acid mediated repression of CYP7A expression. Typically, the animal is a mammal, however, any animal which express cytokines or CYP7A, or repression of CYP7A expression caused by bile acids is included. Particular examples of mammals include humans and non-human primates (apes, macaques, chimpanzees, orangutans, etc.); domesticated animals such as dogs and cast; livestock such as cows, goats, sheep, pigs, horses; and laboratory animals such as rodents (e.g., mice and rats), guinea pigs, and rabbits

Subjects treatable with the methods of the invention include those in need of such treatment due to abnormal or undesirable liver cytokine production or decreased or insufficient CYP7A expression. Such subjects may suffer from a physiological condition or pathological disorder in which treatment with a method of the invention can be beneficial. For example, a subject suffering from liver inflammation caused by a liver insult such as hepatitis or a toxin, such as alcohol poisoning is a candidate subject. Similarly, a subject having elevated LDL-cholesterol (e.g., for human males, greater than about 200 mg/ml) or decreased HDL (e.g., less than about 35 mg/ml) is a candidate subject for treatment.

Subjects treatable with the methods of the invention also include those that do not exhibit abnormal or undesirable liver cytokine production or decreased or insufficient CYP7A expression. However, preventing or inhibiting liver cytokine production or increasing CYP7A expression may be desired in such subjects. For example, a subject at risk of an insult to the liver, such as exposure to hepatitis or a toxin or acute alcohol poisoning can be treated prior to exposure to the insult in order to decrease or prevent inflammation caused by cytokine production following exposure to the insult. Subjects also include apparently normal subjects that do not exhibit overt symptoms but may be at risk, for example, a subject having a family history or genetic predisposition towards excess liver cytokine production or elevated cholesterol.

Candidate subjects may also be identified by screening for the specific insult or disorder, such as exposure to or infection by hepatitis A, B or C; acute or chronic alcohol or tylenol poisoning; cirrhosis; cholestatic liver disease; Reye's syndrome or chemical; or other xenobiotic poisoning. Candidate subjects that produce excess or undesirable amounts of cytokine can be identified using blood tests for specific cytokines associated with liver function tests (e.g. plasma levels of bile acids, serum glutamate pyruvate transaminase (SGPT) and biliruben). Candidate subjects having or at risk of having elevated LDL, decreased HDL, or artherosclerotic lesion formation and artherosclerosis can be identified using well known methods available in the art. For example, genetic screening can identify a subject that lacks a functional LDL receptor or deficient LDL receptor expression which are predisposed to early onset of coronary heart disease.

Treatment of a subject generally results in reducing the severity of one or more symptoms of the condition in the subject, i.e., an improvement in the subject's condition or a “therapeutic effect.” Therefore, treatment can prevent or reduce one or more symptoms of the condition, inhibit progression or worsening of the condition, and in some instances, reverse the condition. Thus, in the case of inhibiting or reducing cytokine production in a subject, for example, treatment optimally reduces levels of one or more cytokines so that chronic or acute liver inflammation or resulting tissue damage is either prevented, reduced, inhibited, arrested (worsening of inflammation or tissue damage is prevented) or reversed (e.g., due to tissue regeneration). Improvement of an inflammatory condition of the liver includes any one of the conditions associated with liver cytokine production described herein or otherwise known in the art. Particular examples include preventing, inhibiting, reducing or arresting liver inflammation or tissue damage, for example, caused by acute or chronic insult from hepatitis A to E; toxin exposure (e.g., alcohol or tylenol or other xenobiotic chemical poisoning such as drugs used in association with organ or tissue transplantation or cleaning agents containing carbon tetrachloride, carbon dichloride, etc.), development of liver cirrhosis or fatty liver. Additional examples include preventing, inhibiting, reducing or arresting liver inflammation or tissue damage that occurs in association with cholestatic liver disease or Reye's syndrome, jaundice, fatty liver and graft vs. host disease (e.g., transplanted liver rejection), necrosis and hypertension (e.g., portal hypertension).

In the case of increasing CYP7A expression or inhibiting bile acid mediated repression of CYP7A expression in a subject, improvement can include, for example, a decrease in levels of cholesterol, LDL, VLDL, triglycerides, or fatty acids or an increase in HDL levels, etc. Improvement of a condition associated with excess or undesirable levels of LDL-cholesterol VLDL, triglycerides, or fatty acids or decreased HDL levels includes any one of the conditions associated with and pathologies resulting from excess or undesirable LDL-cholesterol VLDL, triglycerides, or fatty acids or decreased HDL levels described herein or otherwise known in the art. Particular examples include decreasing the risk of developing coronary heart disease, decreasing or delaying formation of artherosclerotic lesions or artherosclerosis or reducing their severity, decreasing intimal thickening of a blood vessel, reducing risk of coronary heart disease or stroke, cardiac ischemia, peripheral vascular disease, dyslipidemia, hypertension, etc.

The term “ameliorate” means an improvement in the subject's condition, a reduction in the severity of the condition, or an inhibition of progression or worsening of the condition. A subject need not exhibit complete ablation of the condition in order to be beneficial. Thus, amelioration can occur when improvement is incomplete or the desired effect is not completely achieved but is otherwise altered to benefit the host. For example, although a reduction of one or more cytokines in liver may not result in the complete ablation of liver inflammation, or a complete ablation of tissue damage, inhibition or of further inflammation or preventing a worsening of inflammation is still a satisfactory clinical endpoint.

The doses or “sufficient amount” for treating a subject are sufficient to ameliorate one, several or all of the symptoms of the condition, to a measurable or detectable extent although, as discussed, preventing or inhibiting a progression or worsening of the disorder or condition, or a symptom, is a satisfactory outcome. Thus, in the case of a method for treating excess or undesirable cytokine production, a detectable reduction in production of at least one cytokine can be sufficient to ameliorate the condition. Similarly, in the case of a method for treating excess or undesirable LDL-cholesterol or decreased HDL or CYP7A expression levels, a detectable reduction in cholesterol, LDL, VLDL, triglycerides, or fatty acids (e.g. 10% to 20% or more reduction), or increase in CYP7A expression or HDL levels (e.g. 10% to 20% or more increase), can be sufficient to ameliorate the condition. A sufficient amount can be ascertained by measuring the relevant physiological effect or indicator (e.g., cytokines, CYP7A, cholesterol, triglycerides, fatty acids, LDL, VLDL, HDL, etc.). Amounts will also depend upon the condition treated and the therapeutic effect or clinical outcome desired (greater or less, or targeting for a specific effect, e.g. decreasing cholesterol levels without significantly decreasing cytokine production). The skilled artisan will appreciate the various factors that may influence the dosage and timing required to treat a particular subject, including but not limited to the general health, age or gender of the subject, severity of the disease or disorder, previous treatments, etc.

PPARγ agonist and antagonist dosage ranges will typically be from about 0.001 to about 50 mg/kg body weight, or 0.01 to about 20 mg/kg body weight, or 0.1 to about 10 mg/kg body weight. PPARγ agonist dosage ranges for use in the methods of the invention are also described, for example, in Physicians' Desk Reference (1999) 53^{rd} ed., Medical Economics Company, Inc., Montvale, N.J.

PPARγ agonist and antagonists used in the methods of the invention can be formulated into pharmaceutical formulations appropriate for internal or external administration. The pharmaceutical formulations will be in a “pharmaceutically acceptable” or “physiologically acceptable” form. As used herein, the terms “pharmaceutically acceptable” and “physiologically acceptable” refer to carriers, diluents, excipients, and other preparations that can be administered to a subject, without destroying activity or adsorption of the composition.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

PPARγ agonist and antagonists can be incorporated into capsules, particles or a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. Microcapsules can be prepared by coacervation techniques or by interfacial polymerization, for example, by the use of hydroxymethylcellulose or gelatin-microcapsules, or poly (methylmethacrolate) microcapsules, respectively, or in a colloid dispersion system. Colloidal dispersion systems include macromolecule complexes, nano-capsules, microspheres, beads, and lipid-based systems, including oil-in-water emulsions, micelles, mixed micelles, and liposomes. The use of liposomes for introducing various compositions is known in the art (see, e.g., U.S. Pat. Nos. 4,844,904, 5,000,959, 4,863,740, and 4,975,282). Piperazine based amphilic cationic lipids and cationic lipid systems also are known (see, e.g., U.S. Pat. Nos. 5,861,397 and 5,459,127).

A pharmaceutical formulation can be formulated to be compatible with its intended route of administration. Thus, pharmaceutical formulations include carriers, diluents, or excipients suitable for administration by routes including intraperitoneal, intramuscular, intradermal, subcutaneous, oral and intravenous (e.g., portal vein) administration.

Oral formulations include a pill, syrup or elixir. Oral formulations generally include an inert diluent or an edible carrier. For the purpose of oral therapeutic administration, a composition can be incorporated with excipients and used in the form of tablets, troches, or capsules (hard or soft, e.g., gelatin capsules). The tablets, pills, capsules, troches can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Formulations can also include carriers to protect the composition against rapid degradation or elimination from the body, such as a controlled release formulation, including implants and microencapsulated delivery systems. Tablets may be formulated or coated to delay disintegration or absorption in the gastrointestinal tract for sustained action over a longer period of time. For example, a time delay material such as glyceryl monostearate or glyceryl stearate alone, or in combination with a wax, may be employed.

Solutions or suspensions used for parenteral, intradermal, or subcutaneous application can include the following: a sterile diluent such as water for injection, saline solution, fixed oils, polyethylene glycols, glycerine, propylene glycol or other synthetic solvents; antibacterial or antifungal agents such as benzyl alcohol, parabens, chlorobutanol, phenol, ascorbic acid and thimerosal; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Acids or bases, such as hydrochloric acid or sodium hydroxide can be used to adjust pH. The parenteral preparation can be enclosed in ampules, disposable syringes or multiple dose vials made of glass or plastic.

Pharmaceutical formulations suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Isotonic agents, for example, sugars, polyalcohols such as mannitol, sorbitol, sodium chloride can be included in the composition. Prolonged absorption of injectable formulations can be achieved by including an agent that delays absorption, for example, aluminum monostearate or gelatin.

Systemic or localized (e.g., targeted) routes of administration methods and compatible formulations are included. Systemic administration can be achieved, inter alia, by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, compositions can be formulated into ointments, salves, gels, or creams as generally known in the art.

Targeted administration can be achieved by injection or an implantable device located in or near the target cells, tissue or organ (e.g., liver). Targeted delivery can also be achieved by administering via an endoscope, cannula, intubation tube, or catheter. Such devices are also useful for delivering a PPARγ agonist or antagonist to liver. Injection into the portal vein or hepatic artery of the liver is another way in which to achieve local delivery. For example, the formulation can be administered by infusion into the liver over time or a bolus via the portal vein.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

Pharmaceutical formulations including PPARγ agonist and antagonists can include other drugs, therapeutic agents and herbal medicines. Such additional drugs, therapeutic agents and herbal medicines can provide an additive or synergistic effect when used in combination with a PPARγ agonist or antagonist.

As used herein, the terms “drug,” “agent,” or “medicine” are used interchangeably and include any molecule, natural or synthetic, having a biological activity including, for example, small organic molecules, herbal mixtures (e.g., purified and crude extracts), radioisotopes, polypeptides (growth factors, signaling molecules, receptors, antibodies, receptor ligands, etc.), peptidomimetics, nucleic acids (coding for polypeptide or antisense) or fragments thereof. Organic drugs or agents often comprise cyclical carbon or heterocyclic structures, and/or aromatic or polyaromatic structures substituted with one or more functional groups. Drugs or agents are also found among biomolecules, including, but not limited to, saccharides, fatty acids, hormones, vitamins, steroids, purines, pyrimidines, derivatives, structural analogs, or combinations thereof. Known pharmacological drugs and agents are also included (See, for example, Physicians' Desk Reference (1999) 53^{rd} ed., Medical Economics Company, Inc., Montvale, N.J.; and The Pharmacological Basis of Therapeutics, J. G. Hardman and L. E. Limbird, eds. (1996) Ninth ed., McGraw-Hill, New York).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “a cytokine” includes a plurality of cytokines and a reference to “a cell of the liver” includes reference to one or more such cells, and so forth.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLE 1

This example describes methods used for various analysis. This example also describes in vitro and in vivo assays for physiological effects produced by PPARγ agonist activity.

Mouse Studies

Female C3H/HeJ and C57BU16 mice 10-12 weeks old (Jackson Laboratory, Bar Harbor, Me.) were housed in a room with a normal light cycle (lights on from 0600 to 1800) fed water ad libitum with either normal Purina breeder chow or ground Purina breeder chow supplemented with 20% olive oil, 2% cholesterol and 0.5% taurocholic acid (bile acid-contaning atherogenic diet) and water ad libitum. Mice were maintained on this diet for 3 weeks. After 3 weeks, mice were sacrificed at 0900.

To study the effect of rosiglitazone on CYP7A1 expression, C57BL/6 mice fed the chow diet and the bile acid-containing atherogenic diet were divided into two groups. Half the mice in each diet group were given either vehicle (0.25% Tween 80/1% carboxymethylcellulose) alone or vehicle containing 1 mg/ml rosiglitazone daily by oral gavage.

Mice were sacrificed at 0900 and blood was obtained for subsequent analysis. Livers were extracted for RNA, and polyA RNA was isolated, as described (Dueland, et al., 1993, J. Lipid.Res. 34:923-931; Dueland, et al., 1997, J. Lipid Res. 38:1445-1453). Poly A MRNA was blotted onto nitrocellulose and probed with ³²P-labeled cDNA encoding rat CYP7A1 and GAPDH (Duelandet al., 1993, J. Lipid Res. 34:923-931; Dueland, et al., 1997, J. Lipid Res. 38:1445-1453). The relative abundance of CYP7A1 mRNA to GAPDH mRNA was determined using Phosphorimager quantitation (Molecular Biosystems).

HDL cholesterol levels of blood were determined, as previously described (Dueland, et al., 1993, J. Lipid Res. 34:923-931; Dueland, et al., 1997, J. Lipid Res. 38:1445-1453).

Hepatic cytokine mRNAs were quantitated using RNase protection assays, as follows. In vitro transcribed [α-³²P]-UTP labeled -antisense cytokine probes were generated using cytokine multi-probe template kits: Mouse mCK-2 (catalog # 45002P) and Mouse mCK-3 (catalog #45003P) (PharMingen International) and a MAXIscript in vitro transcription kit (catalog #1314) using T7 RNA polymerase per manufacturer instructions. The radiolabeled probes were eluted through G25 Sephadex columns (Boehringer Mannheim) to remove unincorporated nucleotides. RNase protection assays were performed with HybSpeed RPA kits (catalog #1412) (Ambion Inc.) according to the manufacturers specifications. For each reaction, 20 fig of total RNA was hybridized to approximately 50,000 cpm of the antisense cytokine probes and digested with a mixture of RNase A and RNase T1. The protected RNA fragments were separated via a 5% denaturing acrylamide gel. PhosporImager (Molecular Dynamics) analysis was used to visualize the protected RNA fragments.

Cell Culture Studies

Rat L35 cells were cultured in Dulbecco's modified eagle medium (DMEM) as described (Trawick, et al., 1996, J. Lipid Res. 37:24169-24176; Trawick, et al., 1997, J Biol. Chem. 272:3099-3102). THP-1 cells, were cultured as described (Moulton, et al., 1992, Proc. Natl. Acad. Sci. USA. 89:8102-8106).

To examine the effects of conditioned media from THP-1 cells on the expression of CYP7A1 by L35 cells, THP-1 cells were incubated for 48 h RPMI medium 1640 plus 10% FBS and: 0.1% BSA; 0.1% BSA containing CDCA (100 μM); 0.1% BSA containing CDCA. RNA was isolated, blotted onto nitrocellulose and probed with ³²P-labeled cDNA encoding rat CYP7A1 and β-actin. The relative abundance of CYP7A1 mRNA to (β-actin MRNA was determined using Phosphorimager quantitation (Molecular Biosystems).

To examine the effect of bile acids and rosiglitazone on the expression of cytokine mRNAs by THP-1 cells, THP-1 cells were incubated for 48 h RPMI medium 1640 plus 10% FBS containing and: 0.1% BSA; 0.1% BSA containing CDCA (100 ftM); 0.1% BSA containing CDCA and rosiglitazone (500 nM) and 0.1% BSA containing UDCA (100 μM). Cells were harvested and polyA mRNA extracted, as described above. The content of human cytokines mRNAs were quantited by RNase protection assays, as described above except human template kits were used (Human hCK-2-catalog # 45032P and Human hCK-3-catalog # 45033P) (PharMingen International).

Transient Transfections ofPromoter-Luciferase Reporters

Transfections were performed by lipofection under optimized conditions. Typically, L35 cells were transfected with 600 ng promoter constructs (FXR-luc or LXR-luc) with 20 ng of either CMV-LXRα or CMV-FXR (Makishima, et al., 1999, Science. 284 (5418):1362-1365), as indicated. Plasmids were transfected into cells using 4 μl Lipofectamine (Life Technologies) per well in a 12-well plate according to the manufacturer's instructions. Transfection efficiencies were normalized by co-transfecting with pRL-CMV (Promega), a control vector containing a sea pansy (Renilla reniformis) luciferase gene driven by a CMV promoter in the ratio of 1:100. Transfected cells were maintained for 24 h before lysis for reporter assays using the Dual Luciferase Kit (Promega).

Statistical Analysis

Results are given as mean ±S.D. Statistical significance was determined by Students't test using double-tailed p values.

### EXAMPLE 2

This example describes data showing hepatic CYP7A1 mRNA repression in atherosclerosis susceptible C57BL/6 mice fed the atherogenic diet but did not repress CYP7A1 expression in resistant mice. This example also describes data indicating that bile acids increase cytokine expression in vivo in liver.

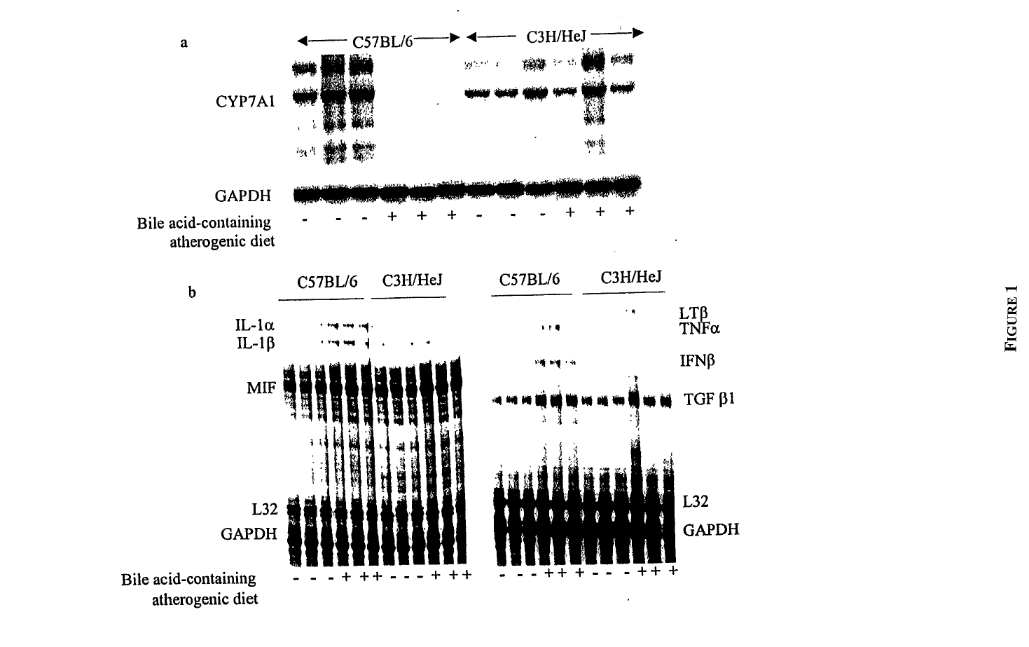

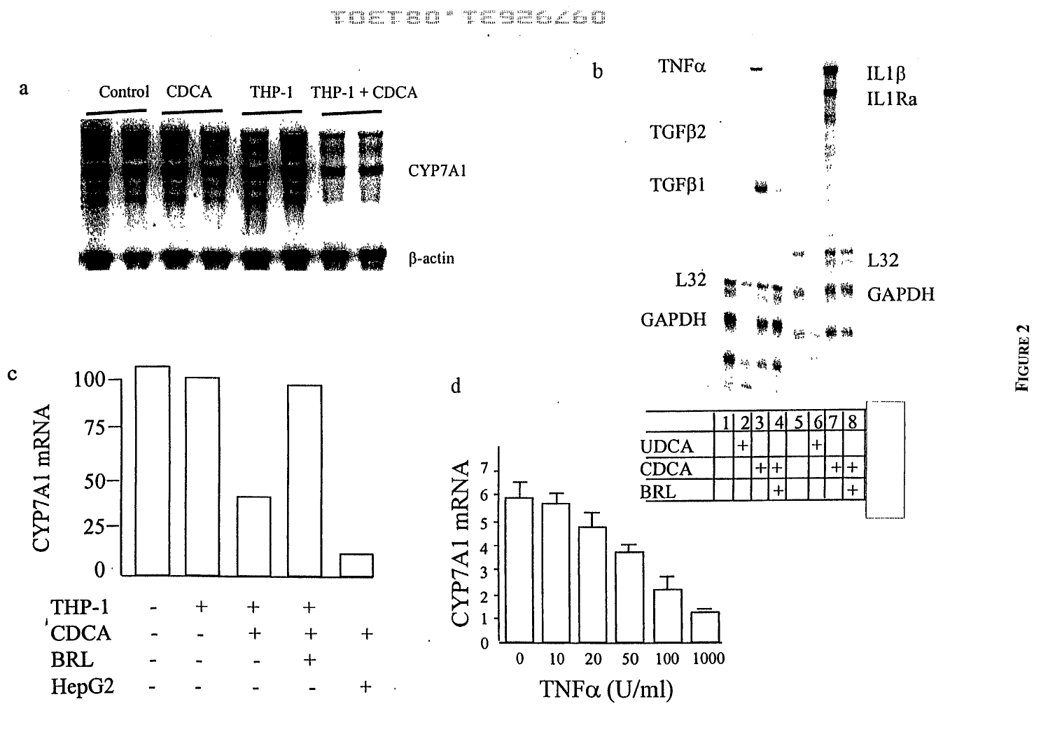

C57BL/6 mice, divided into two groups, were fed the chow diet or the bile acidcontaining atherogenic diet as described in Example 1. The bile acid-containing atherogenic diet markedly decreased hepatic CYP7A1 MRNA expression in atherosclerosis susceptible C57BL/6 mice (70% decrease, p<0.01), whereas it did not repress CYP7A1 expression in atherosclerosis resistant C3H/HeJ mice (FIG. 1A). Moreover, the bile acid-containing atherogenic diet increased the hepatic expression of IL-1α. (7fold, p<0.01), IL-10 (4-fold, p<0.01), TNFα (3-fold, p<0.01), IFNβ (6-fold, p<0.01), and TGF-β1 (7-fold, p<0.01) mRNAs in susceptible C57BL/6 mice, but not in resistant C3H/HeJ mice (FIG. 1B). These findings indicated that activation of regulatory cytokines by liver cells such as resident macrophages or monocytes initiates bile acid negative feedback regulation of CYP7A1.

### EXAMPLE 3

This example describes data indicating that THP cells exposed to bile acid chenodeoxycholic acid (CDCA) increase expression of cytokines, and that rosiglitazone can block CDCA induction of cytokines. This example also describes data indicating that cytokines, induced by bile acid CDCA repress CYP7A1 expression.

Following active absorption in the distal intestine bile acids cross the sinusoidal surface in order to enter the hepatic parenchymal cell (Love, et al., 1998, Curr Opin Lipidol. 9:225-229). Hepatic macrophages (i.e., Kupffer cells) residing at the sinusoidal interface sense bile acids transported across the sinusoids and in response activate expression of cytokines. To approximate the intercellular relationship between hepatic macrophages and parenchymal cells, cultured human monocyte/macrophages (THP-1 cells) were exposed to bile acids and the effects of the conditioned medium examined on the expression of CYP7A1 by L35 cells.

Rat hepatoma L35 cells cultured in serum free medium containing 100 μM of dexamethasone were treated with 0.1% BSA, 0.1% BSA containing CDCA (100 μM) followed by the addition of 50% by volume of medium from THP-1 cells which were incubated for 48 h with 0.1% BSA (THP-1) or 0.1% BSA containing CDCA (100˜LM) (THP-1+CDCA). After 24 hours, cells were harvested, polyA RNA was isolated, blotted onto nitrocellulose and probed with ³²P-labeled cDNA encoding rat CYP7A1 and β-actin.

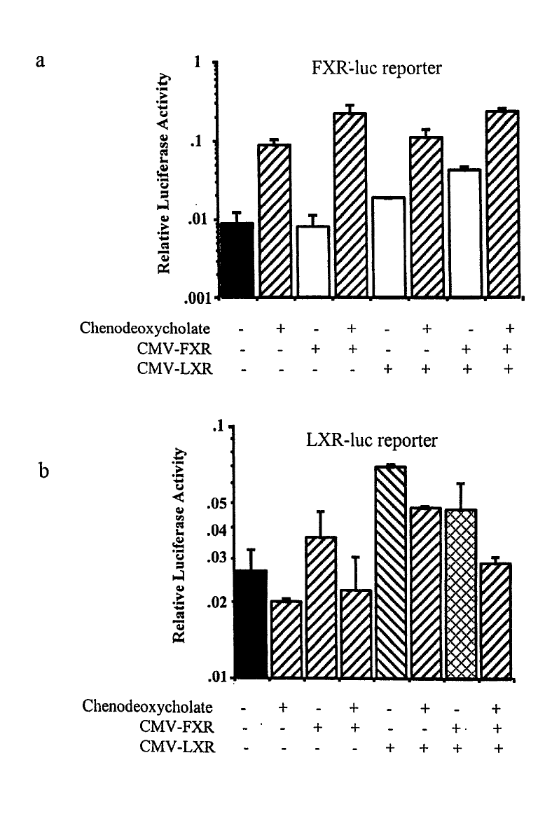

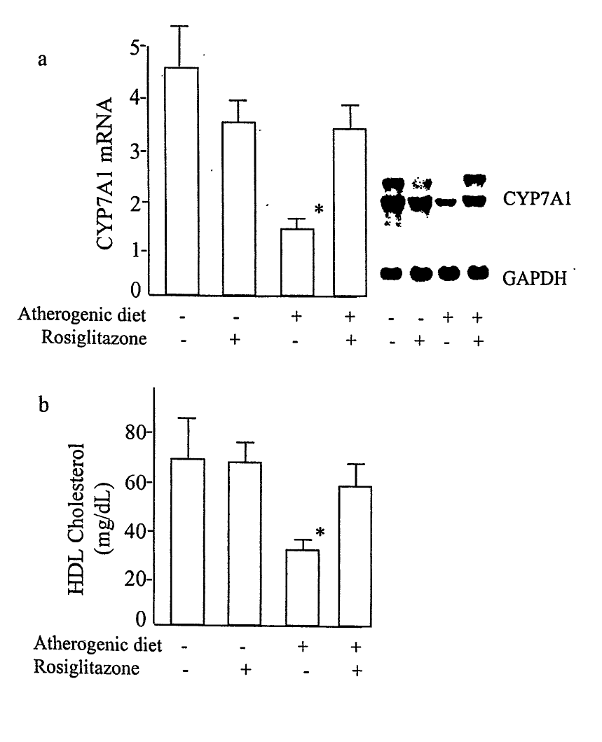

CYP7A1 expression by L35 cells was unaffected by CDCA and the conditioned medium obtained from THP-1 cells (FIG. 2A). However, conditioned medium obtained from THP-1 cells exposed to CDCA repressed CYP7A1 expression by >70% (FIG. 2A). The data indicate that: (1) CDCA requires THP-1 cells in order to repress CYP7A1 expression by L35 cells and (2) CDCA stimulated THP-1 cells to secrete a factor that repressed CYP7A 1.

THP-1 cells incubated with CDCA displayed a marked (>10-fold) induction of TNFα, TGF-β1 and IL1β mRNAs (FIG. 2B). In contrast, the hydrophilic bile acid, UDCA, which does not repress CYP7A1 (Heuman, et al., 1989, J. Lipid Res. 30:1161-1171.) did not induce cytokine expression by THP-1 cells (FIG. 2B).

PPARγ agonism inhibits production of inflammatory cytokines by peripheral monocyte/macrophages in vitro (Jiang, et al., 1998, Nature. 391 (6662):82-86). Treating THP-1 cells with PPARγ agonist rosiglitazone completely blocked the ability of CDCA to induce cytokine mRNAs expression by THP-1 cells (FIG. 2B).

Rat hepatoma L35 cells cultured in serum free medium containing 100 μM of dexamethasone were treated as indicated (FIG. 2C) with conditioned medium obtained from either THP-1 cells incubated with 0.1% BSA (THP-1) containing as designated CDCA (100 μM) and/or rosiglitazone (BRL) or from HepG2 cells incubated with 0.1% BSA containing CDCA (100 μM). After 24 h, cells were harvested and the relative level of CYP7A mRNA to β-actin MRNA was quantitated. The results indicate that rosiglitazone also blocked the ability of THP-1 cells exposed to CDCA to produce conditioned medium that could repress CYP7A1 expression by L35 cells (FIG. 2C). This result indicates that cytokines are responsible for CYP7A1 repression. These findings also show a striking concordance between the ability of different bile acids to induce cytokine expression by THP-1 cells and the ability of the conditioned medium to repress the expression of CYP7A1 InRNA by L35 cells.

HepG2 cells are a human hepatoblastoma cell line that produce multiple cytokines (Stonans, et al., 1999, Cytokine. 11 (2):151-156), whereas L35 cells, do not express detectible levels of cytokine mRNAs. L35 cells were cultured in serum free DMEM medium containing dexamethasone (100 μM) and treated with human TNFa for 24 h and examined for CYP7A1 expression.

Similar to THP-1 cells, HepG2 cells incubated with CDCA produced conditioned medium that repressed CYP7A1 expression by L35 cells (FIG. 2C). This result indicates that liver hepatocytes produce cytokines in response to bile acids. Additional studies showed that TNFa by itself repressed the expression of CYP7A1 by L35 cells (FIG. 2D).

### EXAMPLE 4

This example describes data indicating that rosiglitazone blocks repression of CYP7A1 and reduction of HDL induced by bile acids in animals.

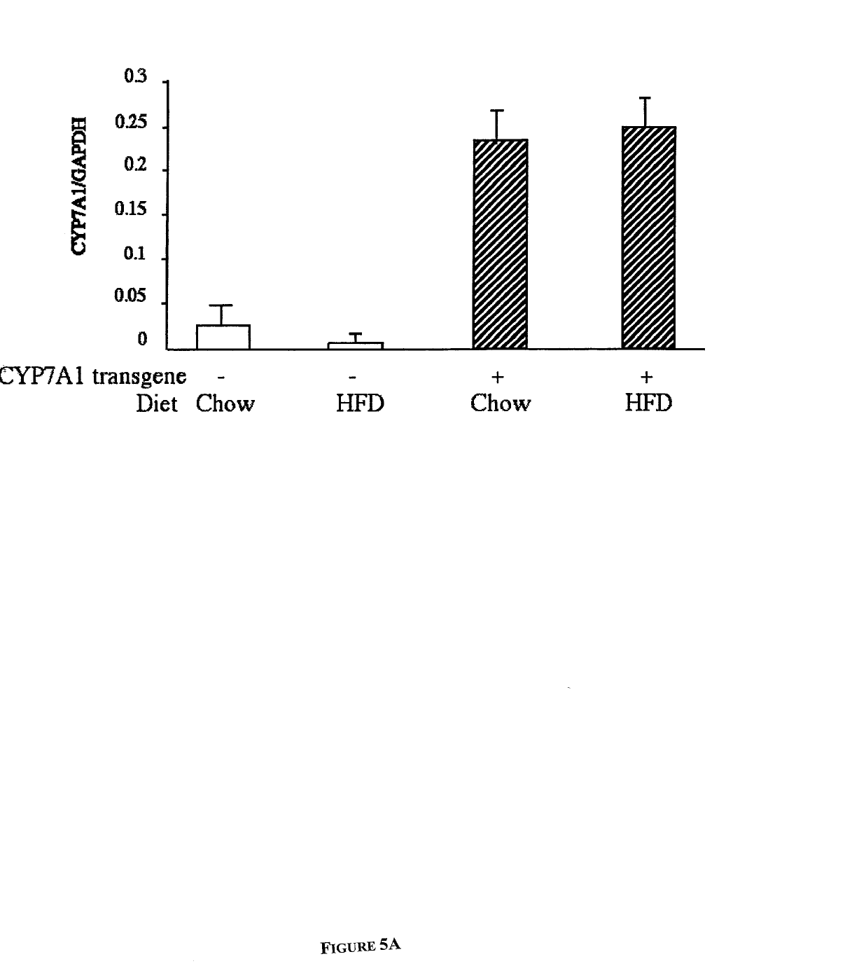

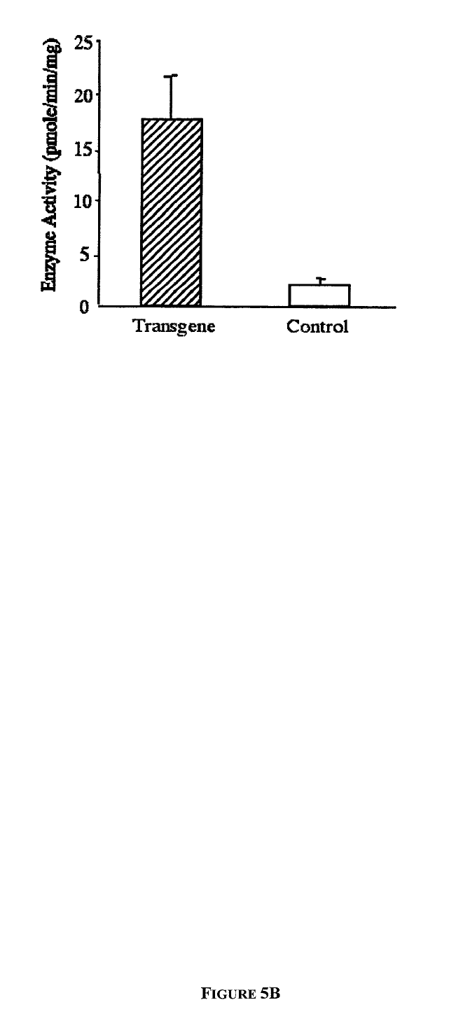

Activation of hepatic cytokines might be the basis for the hepatic inflammation (Liao, et al., 1993, J Clin Invest. 91 (6):2572-2579; Liao, et al., 1994, J Clin Invest. 94 (2):877-884), repression of CYP7A1 (Dueland, et al., 1993, J Lipid Res. 34:923-931; Machleder, et al., 1997, J Clin Invest. 99 (6):1406-1419) and reduction in plasma HDL cholesterol (Dueland, et al., 1997, J. Lipid Res. 38:1445-1453; Machleder, et al., 1997, J Clin Invest. 99 (6):1406-1419; Shih, et al., 1996, J Clin Invest. 97 (7):1630-1639) observed in atherosclerosis-susceptible C57BL/6 mice. To determine if rosiglitazone could block the repression of CYP7A1 and reduction in HDL cholesterol caused by feeding C57BL/6 mice the bile acid-containing atherogenic diet (FIG. 4A) mice were fed the bile acid-containing atherogenic diet with and without rosiglitazone.

Rosiglitazone treatment of chow-fed mice caused a slight 30% decrease (FIG. 4A, p=ns) in the expression of CYP7A1. These data indicate that PPARγ agonism does not directly induce CYP7A1 expression. In contrast, rosiglitazone treatment of mice fed the bile acid-containing atherogenic diet blocked repression of CYP7A1 (i.e., the expression of CYP7A1 mRNA in rosiglitazone-treated mice was not significantly altered by the bile acid-containing atherogenic diet; FIG. 4A).

The effect of rosiglitazone on HDL cholesterol levels was next determined. Rosiglitazone by itself did not affect plasma HDL cholesterol levels in mice fed the chow diet (FIG. 4B). However, adding rosiglitazone to the bile acid-containing atherogenic diet prevented most of the diet-induced decrease in HDL (FIG. 4B).

The results demonstrating that feeding a bile acid-containing atherogenic diet led to a 70% reduction in the expression of hepatic cholesterol-7α-hydroxylase MRNA and increased hepatic expression of cytokines (including TNFα and IL1, known to repress cholesterol-7α-hydroxylase expression) in C57BL/6 mice, but not in C3H/HeJ mice, led to studies which indicated that bile acid negative feedback repression of cholesterol-7α-hydroxylase expression by hepatic parenchymal cells is mediated by cytokines. Studies supporting this conclusion are 1) Incubating human monocyte/macrophage THP-1 cells with chenodeoxycholic acid (CDCA) induced expression of regulatory cytokines as well as produced conditioned medium that when added to rat L35 hepatoma cells caused a marked (70%/o) repression of cholesterol-7α-hydroxylase; 2) PPARγ agonist rosiglitazone, which blocks cytokine production by macrophages in vitro, blocked the CDCA induction of cytokines by THP-1 cells and the production of conditioned medium that repressed cholesterol-7α-hydroxylase expression by L35 cells; and 3) in vivo studies showing that in atherosclerosis-susceptible C57BL/6 mice rosiglitazone blocks bile acid-repression of hepatic cholesterol-7α-hydroxylase expression and decreases plasma HDL levels.

In sum, the aforementioned Examples indicate that 1) PPARγ agonists such as rosiglitazone inhibit activation of cytokine production in liver of animals; and 2) that PPARY agonists such as rosiglitazone inhibit repression of CYP7A1 mediated by bile acids in animals. These conclusions are compatible with those of previous studies suggesting that bile acid negative feedback acts by inhibiting CYP7A1 gene transcription (Pandak, et al., 1991, J. Biol. Chem. 266:3416-3421; Twisk, et al., 1993, Biochem J. 290 (3):685-691) via activating protein kinase C (Stravitz, et al., 1995, J. Lipid Res. 36:1359-1369) as well as FXR (Makishima, et al., 1999, Science. 284 (5418):1365-1368; Stravitz, et al., 1995, J Lipid Res. 36:1359-1369). The production of cytokines by HepG2 cells and the presence of cytokine producing cells (i.e., Kupffer cells and endothelial cells) in preparations of primary rat hepatocytes can explain the ability of CDCA to directly repress CYP7A1 in these experimental models.

### EXAMPLE 5

This example describes data indicating that constitutive high level expression of a CYP7A1 transgene in atherosclerosis-susceptible C57BL/6 mice prevents reduced HDL levels and atherosclerosis lesion formation.

To determine whether CYP7A1 was the causative factor responsible for the parallel changes in hepatic LDL receptor mRNA expression and plasma levels of HDL, a CYP7A1 transgene was expressed in atherosclerosis susceptible C57BL/6 mice. Transgenic mice were produced by injecting the nuclei of blastocysts with a transgene constructed using the liver specific enhancer obtained from the human apo E promoter element. The blastocysts were subsequently implanted into pseudo-pregnant mothers and the progeny displaying the transgen were used for further breeding.

CYP7A1 transgenic mice and non-transgenic control mice were fed a “high-fat” diet containing taurocholate for six weeks. Plasma was isolated retro-orbitally at midlight after an overnight (˜16 h) fast.

While feeding mice the atherogenic diet containing taurocholate decreased the expression of the endogenous CYP7A1 MRNA in non-transgenic littermates, expression the CYP7A1 transgene MRNA was not repressed (FIG. 5A). As a result, on the atherogenic diet the expression of CYP7A1 mRNA was 20-fold greater in CYP7A1 transgenic mice (FIG. 5A). Moreover, hepatic microsomes obtained from transgenic mice fed the atherogenic diet displayed ˜20-fold greater enzymatic activity of CYP7A1 compared to non-transgenic littermates (FIG. 5B). The ability to prevent the decrease in CYP7A1 expression caused by the atherogenic high-fat diet by expressing a CYP7A1 transgene indicated that C3H/HeJ strain-specific resistance to repression of CYP7A1 is responsible for both the resistance to decreased HDL and atherosclerosis caused by the atherogenic “high-fat” diet.

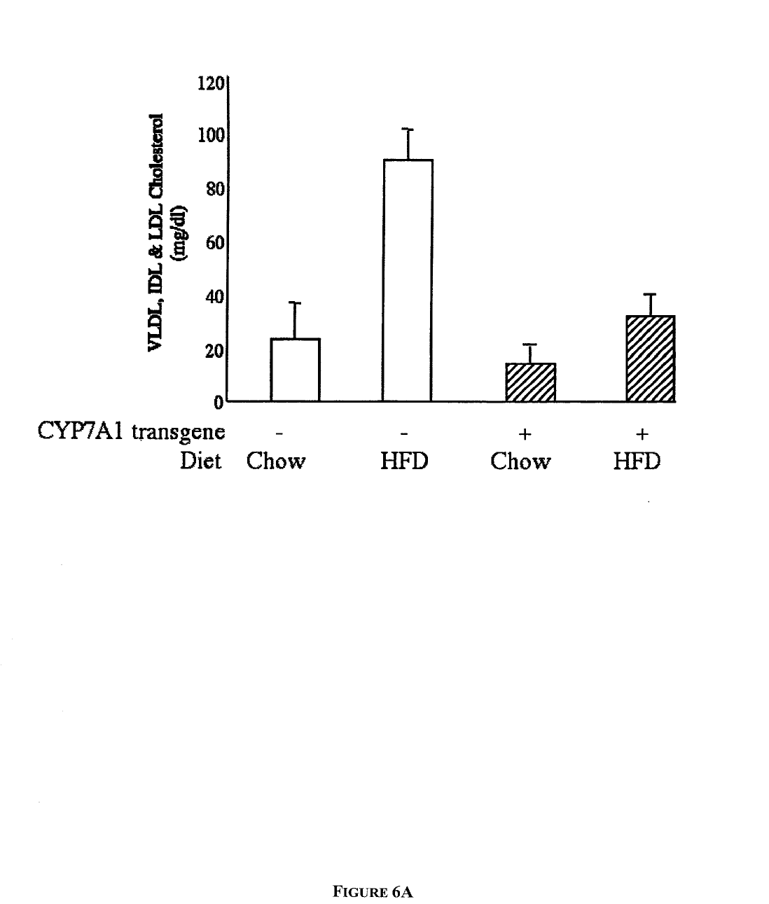

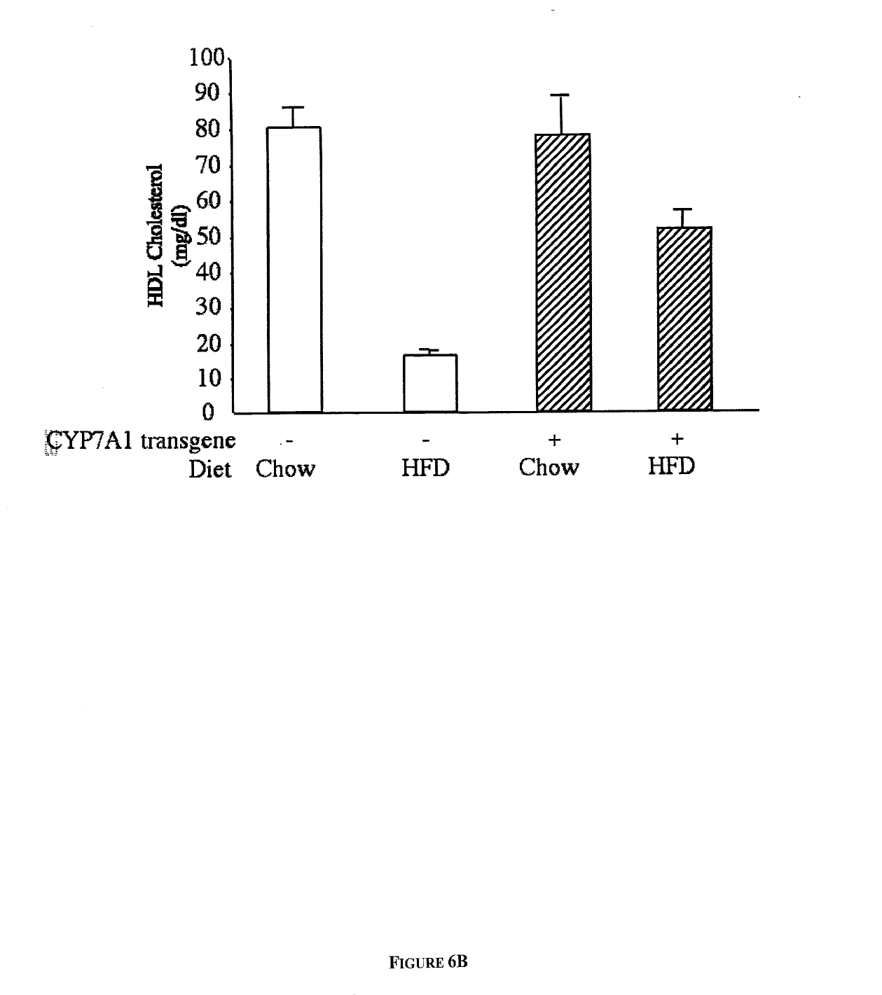

Animals were also analyzed for plasma levels of cholesterol, VLDL, IDL and LDL. On a chow diet, transgenic mice displayed a 50% reduction in the plasma levels of cholesterol in the lipoprotein particles containing apo B (i.e. VLDL, IDL and LDL; FIG. 6A). Feeding the CYP7A1 transgenic mice the atherogenic diet increased VLDL, IDL and LDL cholesterol by 2-fold, reaching the levels displayed by non-transgenic mice fed the chow diet (FIG. 6A). Non-transgenic littermates displayed increased susceptibility to diet-induced hypercholesterolemia (i.e. the atherogenic diet increased VLDL, IDL and LDL cholesterol by 4-fold; FIG. 6A). Thus, on the atherogenic diet, plasma levels of VLDL, IDL and LDL cholesterol, in nontransgenic littermates were 5-fold greater than in CYP7A1 transgenic mice.

The results indicate that CYP7A1 transgenic mice displayed a remarkable resistance to reduced HDL levels in response to the atherogenic diet. On the chow diet plasma HDL cholesterol levels were similar in CYP7A1 transgenic and non-transgenic littermates (FIG. 6B). When fed the atherogenic diet non-transgenic mice displayed a 50% decrease in plasma HDL cholesterol (FIG. 6B). This 50% reduction in plasma HDL cholesterol levels displayed by non-transgenic littermates was nearly identical to the reduction in HDL cholesterol reported for inbred C57BL/6J mice (Dueland, et al., 1997, J. Lipid Res. 38:1445-1453). In contrast, CYP7A1 transgenic mice displayed resistance to diet reduction in HDL cholesterol; the atherogenic diet caused only a 15% decrease (not statistically significant, p=ns) in plasma HDL cholesterol levels (FIG. 6B). These data therefore demonstrate that the presence of constitutive CYP7A1 expression blocked the ability of the atherogenic diet to reduce plasma HDL cholesterol levels in C57BL/6 mice.

### EXAMPLE 6

This example describes data showing that mice expressing CYP7A1 transgene had fewer atherosclerotic lesions than non-transgenic littermates.

Previous studies indicate that in response to the high-fat” diet inbred C57BL/6 mice develop fatty streak atherosclerosis lesions which can be visualized and quantitated from thin sections obtained from heart valves (Tangirala, et al., 1995, J. Lipid Res. 36:2320-2328; Shih, et al., 1995, Mol Med Today. 1 (8):364-372; Paigen, et al., 1987, Atherosclerosis. 68 (3):231-40). Mice expressing CYP7A 1 transgene were examined for decreased presence of diet-induced atherosclerotic lesions.

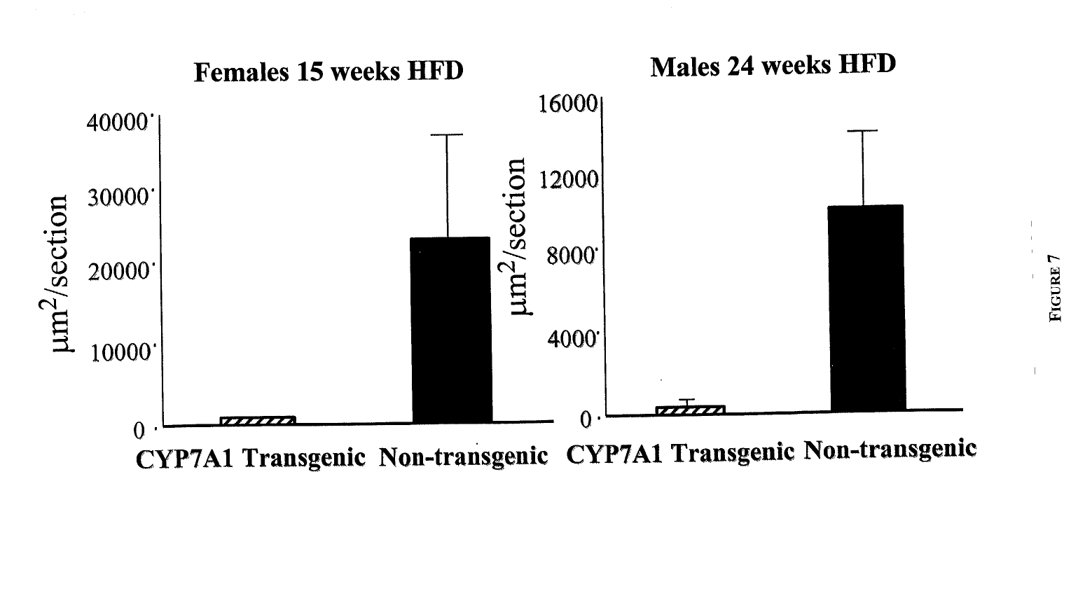

In the first study, female littermate mice from both groups were fed the atherogenic diet for 15 weeks. Plasma lipids levels were identical to those shown after 8 weeks of the diet. Moreover, while non-transgenic mice displayed significant levels of atherosclerotic lesions, CYP7A1 transgenic littermates showed undetectable levels of atherosclerosis (FIG. 7).

To examine if similar results would be obtained in male C57BL/6 mice, which compared to females have a reduced susceptibility to diet-induced atherosclerosis (Shih, et al., 1995, Mol Med Today. 1 (8):364-372) male mice obtained from both groups were fed the atherogenic diet for 24 weeks. Hearts were isolated after the indicated length of time on the “high fat” diet imbedded, thin sectioned and stained with oil-red 0 and analyzed for atherosclerotic lesions. Heart section analysis of female mice fed a high fat diet for 15 weeks (6 transgenic and 7 nontransgenic). Male mice fed the high fat diet for 24 weeks (9 mice in each group)

The analysis revealed that while non-transgenic mice developed significant aortic valve lesions, none of the CYP7A1 transgenic male mice displayed detectable lesions (FIG. 7). The data therefore demonstrate that transgenic expression of CYP7A1 in susceptible C57BL/6 mice recapitulates the atherogenic resistant phenotype exhibited by C3H/HeJ mice. These studies further establish the importance of CYP7A1 in regulating lipoprotein metabolism and susceptibility to atherosclerosis in C57BL/6 mice.

What is claimed is:

1. A method of inhibiting production of a cytokine by a cell of the liver, comprising contacting a cell of the liver that expresses a cytokine with an amount of a PPARγ agonist sufficient to inhibit production of a cytokine by the cell.

2. The method of claim 1, wherein the cytokine is an inflammatory cytokine.

3. The method of claim 2, wherein the cytokine is IL-1α, IL-1β, TNFα, IFNβ, IFNγ or TGF-β1, and the.

4. The method of claim 1, wherein the cell is a Kupffer cell, hepatocyte, bile ductal cell, parenchymal cell or endothelial cell.

5. The method of claim 1, wherein the PPARγ agonist comprises rosiglitazone, or an analogue or derivative thereof.

6. The method of claim 1, wherein the PPARγ agonist comprises a thiazolidinedione.

7. The method of claim 7, wherein the thiazolidinedione is pioglitazone, darglitazone, fluoroglitazone, troglitazone, BRL 49653, ciglitazone, englitazone, AD 5075, a salt thereof or an analogue or derivative thereof.

8. The method of claim 1, wherein the PPARγ agonist comprises a prostaglandin, a fatty acid or a metabolite thereof.

9. The method of claim 1, wherein the contacting is in vivo or ex vivo.

10. A method of inhibiting production of a cytokine in the liver of a subject comprising administering a PPARγ agonist to the subject in an amount sufficient to decrease production of one or more cytokines in the liver.

11. The method of claim 10, wherein the PPARγ agonist comprises rosiglitazone, or an analogue or derivative thereof.

12. The method of claim 10, wherein the PPARγ agonist comprises a thiazolidinedione.

13. The method of claim 12, wherein the thiazolidinedione is pioglitazone, darglitazone, fluoroglitazone, troglitazone, BRL 49653, ciglitazone, englitazone, AD 5075, a salt thereof or an analogue or derivative thereof.

14. The method of claim 10, wherein the PPARγ agonist comprises a prostaglandin, a fatty acid or a metabolite thereof.

15. The method of claim 10, wherein the cytokine is an inflammatory cytokine.

16. The method of claim 15, wherein the cytokine is IL-1α, IL-1β, TNFα, IFNβ, IFNγ or TGF-β1.

17. The method of claim 10, wherein the subject is a human.

18. A method of inhibiting liver damage or susceptibility to liver damage caused by production of a cytokine in the liver comprising administering a PPARγ agonist to a subject in an amount sufficient to inhibit liver damage or susceptibility to liver damage caused by production of the cytokine.

19. The method of claim 18, wherein the PPARγ agonist comprises rosiglitazone or an analogue or derivative thereof.

20. The method of claim 18, wherein the PPARγ agonist comprises a thiazolidinedione.

21. The method of claim 20, wherein the thiazolidinedione is pioglitazone, darglitazone, fluoroglitazone, troglitazone, BRL 49653, ciglitazone, englitazone, AD 5075, a salt thereof or an analogue or derivative thereof.

22. The method of claim 18, wherein the PPARγ agonist comprises a prostaglandin, a fatty acid or a metabolite thereof.

23. The method of claim 18, wherein the cytokine is an inflammatory cytokine.

24. The method of claim 23, wherein the cytokine is IL-1α, IL-1β, TNFα, IFNβ, IFNγ or TGF-β1.

25. The method of claim 18, wherein the subject is a human.

26. A method of treating or reducing the risk of an inflammatory condition of the liver in a subject comprising administering a PPARγ agonist to the subject in an amount sufficient to treat or reduce the risk of the inflammatory condition of the liver.

27. The method of claim 26, wherein the PPARγ agonist comprises rosiglitazone or an analogue or derivative thereof.

28. The method of claim 26, wherein the PPARγ agonist comprises a thiazolidinedione.

29. The method of claim 28, wherein the thiazolidinedione is pioglitazone, darglitazone, fluoroglitazone, troglitazone, BRL 49653, ciglitazone, englitazone, AD 5075, a salt thereof or an analogue or derivative thereof.

30. The method of claim 26, wherein the PPARγ agonist comprises a prostaglandin, a fatty acid or a metabolite thereof.

31. The method of claim 26, wherein the inflammatory condition comprises alcoholic liver disease, cirrhosis, tylenol poisoning, Reye's syndrome, acute or chronic xenobiotic poisoning, acute or chronic hepatitis infection, or cholestatic liver disease.

32. The method of claim 26, wherein the subject is a human.

33. A method of increasing cholesterol-7α-hydroxylase (CYP7A) expression comprising contacting a cell of the liver with an amount of a PPARγ agonist sufficient to increase cholesterol-7α-hydroxylase (CYP7A) expression by the cell.

34. The method of claim 33, wherein the cell is a Kupffer cell or hepatocyte.

35. The method of claim 33, wherein the PPARγ agonist comprises rosiglitazone, or an analogue or derivative thereof.

36. The method of claim 33, wherein the PPARγ agonist comprises a thiazolidinedione.

37. The method of claim 36, wherein the thiazolidinedione is pioglitazone, darglitazone, fluoroglitazone, troglitazone, BRL 49653, ciglitazone, englitazone, AD 5075, a salt thereof or an analogue or derivative thereof.

38. The method of claim 33, wherein the PPARγ agonist comprises a prostaglandin, a fatty acid or a metabolite thereof.

39. The method of claim 33, wherein the contacting is in vivo or ex vivo.

40. The method of claim 33, wherein the cell is present in a subject.

41. The method of claim 40, wherein the cell is human.

42. A method of inhibiting bile-acid mediated repression of cholesterol-7α-hydroxylase (CYP7A) comprising contacting a cell of the liver with an amount of a PPARγ agonist sufficient to inhibit bile-acid mediated cholesterol-7α-hydroxylase (CYP7A) repression.

43. The method of claim 42, wherein the cell is a Kupffer cell or hepatocyte.

44. The method of claim 42, wherein the PPARγ agonist comprises rosiglitazone, or an analogue or derivative thereof.

45. The method of claim 42, wherein the PPARγ agonist comprises a thiazolidinedione.

46. The method of claim 45, wherein the thiazolidinedione is pioglitazone, darglitazone, fluoroglitazone, troglitazone, BRL 49653, ciglitazone, englitazone, AD 5075, a salt thereof or an analogue or derivative thereof.

47. The method of claim 42, wherein the PPARγ agonist comprises a prostaglandin, a fatty acid or a metabolite thereof.

48. The method of claim 42, wherein the contacting is in vivo or ex vivo.

49. The method of claim 42, wherein the cell is present in a subject.

50. The method of claim 49, wherein the cell is human.
